(19) United States
(12) Patent Application Publication
(10) Pub. No.: US 2010/0210011 A1
(43) Pub. Date: Aug. 19, 2010

(54) MARKING EAR TAG COMPRISING A TISSUE SAMPLING DEVICE

(75) Inventors: Jean Jacques Hilpert, Vitre (FR); Johan Decaluwe, Laval (FR)

Correspondence Address: YOUNG & THOMPSON, 209 Madison Street, Suite 500, Alexandria, VA, 22314, US

(73) Assignee: CHEVILLOT SAS, Albi Cedex 9 (FR)

(21) Appl. No.: 12/666,084

(22) PCT Filed: Jun. 17, 2008
(86) PCT No.: PCT/FR08/00841

§ 371 Date: Feb. 4, 2010

(30) Foreign Application Priority Data
Jun. 22, 2007 (FR) 07 04466

(51) Int. Cl. C12M 1/00; A01K 11/00

(52) U.S. Cl. 435/307.1; 40/301

(57) ABSTRACT

An ear tag for identifying animals, enables a sample of biological tissue to be taken when the tag is applied to the animal. The tag includes a male part and a female part which are assembled by irreversible socketing. The stem of the male part includes a detachable head having a hollow distal part provided with a cutting edge for cutting out a biological sample when the head perforates the ear of the animal. The female part includes a central tube for receiving the head and at least partially receiving the stem when the ear tag is applied, the tube being associated with a detachable sample container arranged in the axial extension of the tube. The proximal end of the container has a first opening for receiving the head of the stem when the ear tag is applied, and the distal end of the container has a second opening enabling the inside of the sample container to be reached.

This invention belongs to the field of marking animals using devices that ensure inviolability and traceability.

It relates to an ear tag for identifying an animal that is designed to enable a sample of tissues to be taken by assembly of a device for preserving a biological sample at the moment the tag is applied to the animal. This device, which makes it possible to preserve and later analyze the sample that is taken, is also an object of this invention. The sampling system is designed so that the sample can be easily handled and accessed for the analysis thereof.

In recent years, the identification of livestock such as cattle, sheep or pigs has become mandatory in numerous regions of the world. This is generally carried out by applying an ear tag on one or both ears of the animal, whereby said ear tag bears a number that allows an individual identification of each animal. Numerous types of tags exist, which all comprise a female part or a receiving part, and a male part or a piercing. During the application, the animal's ear is placed between the male part and the female part that are then assembled through the animal's ear by using a suitable clamp. Once in place, the ear tag should be impossible to remove, so as to prevent fraud. For this purpose, a certain number of locking mechanisms have been conceived for allowing a perfect retention of the male part in the female part once the tag has been applied to the animal's ear. For example, the patents EP 0177201, EP 0913081, WO 95/25426 and EP 1037525 describe locking mechanisms that assume the shape of retention rings, retention strips and/or clips that hold the male part in the female part once the tag has been assembled.

Furthermore, it is becoming increasingly important to be able not only to identify an animal by means of the identification number marked on the tag but also to take a sample of biological material from the animal that bears said tag so that it is possible to combine the biological/biochemical characteristics of the sample in question with the animal from which the sample has been taken during a subsequent stage.

The document EP1014861 describes a tool for taking samples of biological material and a capsule that is equipped with a cover. The sample container and the capsule cover are part of an ear tag that is applied at the same time as the sample is introduced into the container and the cover closed on the former. The thus formed test capsule bears the same written information as the thus applied ear tag but can be separated from it. The sample that is contained in the capsule is then sent to a laboratory for the purpose of carrying out tests whose purpose is to determine whether the animal concerned is infected by a certain illness or is immunized.

EP 1088212 describes a tool that is used to take and prepare a sample of biological material to carry out an analysis of molecular biology. The tool is equipped with a sample container and a means of taking samples, means that penetrates the sample container after having imitated the sampling and closes the container in an airtight manner. Inside the sample container are found means for preserving the sample against DNA-degrading enzymes. The means of taking a sample is embedded through the animal's ear and then in the sample container that is itself combined with an ear tag that comprises a plate with a punch and a plate with an opening. The sample container is placed without attachment in the plate with an opening and can therefore be withdrawn once the sample has been taken, whereas the ear tag remains in the animal's ear.

EP 1372379 describes an ear tag for identifying animals with a mechanism for taking biological samples. In this ear tag, the male part is designed in two elements that can be separated. The first of these elements remains in the female part of the ear tag once the former has been applied, whereas the other element is used to take a sample of biological material and can be moved into the first element.

The drawback of the devices that are described in the documents EP1014861 and EP1088212 is that the thus produced samples should be stored in a reliable and airtight manner in the respective test capsules. The analyses to be carried out subsequently by a laboratory require a complicated procedure of opening the secure test capsule. This requires in particular the application of a significant force or the use of equipment that is specific to this operation, which makes the operation long and difficult.

In addition, with the closed capsule that is described by EP 1014861 and EP 1088212, it is not truly possible to see whether a sample has been effectively and correctly collected while the capsule has not been opened. Once it is open, difficult and numerous manipulations are necessary, carried out by an operator or using an exclusive piece of equipment dedicated to these operations alone.

Consequently, one objective of this invention is to propose an ear tag that makes it possible to take a sample of biological material and facilitates the subsequent treatment of said sample. Furthermore, one objective of this invention is to propose an ear tag that makes possible the assembly of a device for taking and preserving a tissue sample that is compatible with the equipment that is commonly used in a laboratory. It is also an objective of this invention to propose a device for preserving a biological sample that makes possible an easier manipulation and that consequently adapts more easily to the different laboratory procedures.

The objectives of this invention are reached using an ear tag for identifying an animal, comprising a male part and a female part that are designed to be assembled by irreversible interlocking on both sides of said animal's ear,
Whereby said male part comprises a base and a stem constituting an extension of said base, said stem having a removable head whose distal part is hollow and is equipped with a cutting edge that can cut a sample of biological material into sections when said head perforates the animal's ear,
Whereby said female part comprises a central tube that can receive said head and at least partially said stem during the application of the ear tag, whereby said tube is combined with a detachable sample container that is placed in the axial extension of said tube, said container having at its proximal end a first opening that can receive the head of said stem during the application of the ear tag,
   whereby said ear tag is characterized in that said container comprises at its distal end a second opening that provides access to the inside space of said sample container.

The inventors have actually demonstrated that by providing an ear tag with a female part that comprises a detachable sample container, which comprises an additional opening, the subsequent manipulation and analysis of the sample are greatly facilitated. The additional opening allows direct work on the sample inside the sample container without having to extract the sample from said container.

According to the invention, the head is removable. It can be attached in a loose manner or simply applied to the stem: it is then placed manually at the top of the stem, before applying the mark on the animal's ear. It can also, in a known manner, be pre-mounted on the stem, with a breaking point between the stem and the head. This breaking point can take the form of a portion of material that is embrittled between the stem and the head, for example using a weaker section. The cutting edge of the head is then the most prominent point of the male part and consequently it is the one that perforates the tissues when the male part is assembled with the female part on the animal's ear, the thus cut-out fragment remaining in the hollow part of said head. This is the reason for which the head represents the true tool for taking samples of biological material. Once the head and the stem of the male part have passed through the animal's ear, the head, by the exerted pressure, penetrates the first opening in the sample container and disengages itself from the stem, as will be explained in detail later.

In one embodiment of the tag according to the invention, the head assumes the shape of a capsule whose proximal part is sealed by a bottom and whose distal part that is equipped with said cutting edge is an open orifice. When the head passes through the animal's ear, the fragment of tissues that is cut into sections is thus collected in the cylindrical part of the head.

In one embodiment of the tag according to the invention, the open orifice of the distal part of the head is placed in the same axis as the sample container. It is therefore placed in the alignment of the former, which makes it possible to easily access the sample that is collected, directly from the second opening, through the container.

The cutting edge, whose edge defines the limit of said open orifice of the distal part of the head, can assume the shape of a circle, an ellipse, a square, a triangle, a rectangle, an octagon or another polygon. The geometry of the edge thus determines the shape and the size of the sample of biological material that is taken.

In one particular embodiment of the tag according to the invention, the orifice of the distal part of the head has a surface area that is greater than that of the second opening of the sample container. The size of the sample is then greater than the second opening of the container, so that the sample of biological material cannot fall from said sample container after it is taken.

In another particular embodiment of the tag according to the invention, the second opening of the sample container and the orifice of the distal part of the head have an identical or approximately identical surface. The opening can then be as large as possible and large enough to allow the analysis of the sample on the inside of the container, for example with the use of reagents on this sample, such as cell lysis reagents, or for the enzymatic treatment of the sample, nucleic amplification, drying, or else for the purpose of heat treatment.

In a preferred embodiment of the tag according to the invention, the inside wall of the proximal end of the container and the outside wall of the distal part of the head have a complementary shape such that said proximal end of the container and said distal part of the head are able to fit together perfectly. This implies that they have the same shape and the same dimension over the length where interlocking is carried out. Thus, when the male part of the tag engages in the central tube of the female part, the end of the stem that bears the detachable head encounters the proximal end of the sample container. The pressure that is exerted causes the insertion of the head into the container through said proximal end. Said head and said proximal end adapt to one another exactly; they fit together perfectly. An additional thrust causes the advance of the stem up to its locking position and the ejection of the container that becomes detached by taking away the head.

The term “perfectly” that is used here refers to the fact that the two pieces fit together thoroughly without leaving space between their walls, a slight pressure sufficient to overcome the forces of friction. In a different embodiment, it is possible, however, to use holding means, such as one or more tabs, which make possible the input of the head into the first opening of the sample container but prevent its exit once it is inside.

In one common embodiment of the tag according to the invention, the head can assume the shape of a cylindrical capsule, and the cutting edge is circular.

Likewise, the sample container can assume the general shape of a cylinder that comprises said first opening at its proximal end and said second opening at its distal end. The first opening and the second opening are thus preferably located at each of the ends of the sample container, whereby the first opening allows the input of the head and consequently also the sample of biological material, whereas the second opening at the opposite end allows a facilitated analysis of the sample.

Said sample container can have a circular straight-line cross-section over its entire length or else over a part of the latter only, whereby the remainder adopts a cross-section of different shape. In particular, the outside wall of the sample container can comprise a flat part that makes it possible to write a code or to affix an identification mark. According to a preferred characteristic of the tag according to the invention, the outside wall of the sample container comprises a flat surface that can receive—or be used as—substrate for an identifying marking.

The ear tag is made of a suitable plastic material, for example, one of those indicated in the documents EP 913803 or EP 0177201. The sample container is also preferably made of plastic, according to the techniques that are known to one skilled in the art, subject to the criteria below. Actually, according to a preferred characteristic of the tag according to the invention, the sample container is at least partially transparent. Preferably, it is transparent at least at its side walls. Of course, there is no reason why it cannot also be transparent at one and/or the other of its ends. This is designed to allow the verification of the presence of a sample of biological material inside said container.

The sample container can be made of a single material that has the desired properties, or else of two different materials that are selected independently. Thus, the sample container can be made of one or more plastic materials that are selected from the group that comprises polymethyl methacrylate, polycarbonate, polypropylene, polyethylene, polymethylpentene, terephthalic polyethylene, polystyrene, polyvinyl chloride and polyvinyl toluene. If a part of said container is transparent, it can be made of, for example, polymethyl methacrylate, polycarbonate, polypropylene, polyethylene, polymethylpentene, or terephthalic polyethylene.

It is understood that the choice of material makes it possible to reconcile, if desired, the requirement of transparency and ease of marking. For example, the proximal end can be transparent while the distal end can be used as a substrate for an identifications means, for example by ink or laser printing. In an alternative variant embodiment, only the second opening, which is found at the distal end, is transparent. In the same way, the head itself can be transparent, or at least partially transparent, for example in a position that makes possible an inspection inside the tube along the longitudinal axis. According to certain embodiments, the head can also have a magnifying glass, making it possible to verify whether or not a sample is present. One skilled in the art will have no problem in making his selection from among the materials that are offered on the market corresponding to these criteria.

Another object of this invention relates to a device that makes possible the preservation, the storage and the manipulation of a biological sample. This device is obtained from constituent elements of the identification tag that was just described, i.e., by assembly of said elements and then ejection during the installation of the ear tag on the livestock.

More specifically, a device for preserving a biological sample taken from the ear of an animal during the application of an identification tag according to the invention is claimed, whereby said device comprises, on the one hand, a sample container that comprises, at its proximal end, a first opening that is sealed by the distal part of a hollow head that contains said biological sample, and, at its distal end, a second opening that provides access to the inside space of said sample container, whereby said proximal end of the container and said distal part of the head fit together perfectly, and, on the other hand, a protection tube that is equipped with a cover that is designed to seal the neck of said tube, whereby said tube can keep the sample container in a closed space.

The protection tube can consist of any receptacle that makes it possible for the sample to be isolated from the outside environment. It is equipped with a cover that is designed to seal the neck of said tube so that the container, in its entirety, is enclosed in the protection tube, itself having closing means. In this way, the sample that is present in the container is confined in the inside space of the protection tube, which promotes the physical protection of the sampling device at the same time as the preservation of the tissue sample that it contains.

According to an advantageous characteristic, the device that is the object of the invention can comprise means for keeping a sample container in suspension at an intermediate height in the tube. An intermediate height is such that the container does not abut against the bottom of the protection tube but is held by a suspension means so that the inside volume of the container (and therefore the sample) stays in connection with the atmosphere of the protection tube.

According to a particular embodiment, said holding means comprise a collar that is located on the outside wall of the container and a shoulder that is located on the inside wall of the protection tube, whereby said collar rests on said shoulder. The relative location of the collar and the shoulder is, of course, adapted so that the container occupies an intermediate position in the tube. It is possible to have, for example, a shoulder in the top part of the tube (close to the neck) and to place the collar at the proximal end of the sample container. In this case, when the sample container is engaged in the tube, its second opening is oriented toward the bottom of the protection tube, as well as the orifice of the hollow head that contains the sample. To carry out the analyses, it will be suitable to recover the container that is combined with the head containing the sample.

In a particularly advantageous implementation variant, the collar is located at the distal end of the outside wall of the sample container, i.e., close to the second opening that makes access possible to the inside volume of the container. The shoulder is in the upper part of the tube (close to the neck). In this configuration, when the sample container is engaged in the protection tube, its second opening is oriented toward the top of the protection tube. The head, which is found in the low part of the tube, has its orifice oriented toward the top: the sample is thus directly accessible from the neck of the tube through the second orifice of the container. It thus is possible to carry out certain operations without extracting the contents of the tube. Furthermore, all of the manipulations, treatments and analyses can be done by simply placing the open tube, or the sample container by itself, in a standard laboratory substrate. A significant advantage of this embodiment is that the sample that is still in the hollow housing of the head does not run the risk of falling from its housing, even if its size has decreased, for example, after drying, contrary to other systems where the device is to be reversed during the manipulations. The collar optionally acts as a holding means in the laboratory substrate.

According to an advantageous characteristic of the device that is the object of the invention, the protection tube can contain at least one chemical or biochemical compound that is useful for the treatment of the sample. “Chemical or biochemical compound that is useful for the treatment of the sample” is defined as both a compound in solid form and in solution, whereby this compound can have a preservation action or play a role for preparing the sample for an analysis, or even be a reagent that is necessary for a subsequent analysis.

According to an advantageous characteristic of the invention, the protection tube contains at least one chemical or biochemical compound that is selected from among the drying agents, the protease inhibitors, the enzymes, the DNA fragments, and the RNA fragments. For example, the protection tube can contain, even before the introduction of the sample containers, chemical agents or solutions of reagents that are useful for the subsequent treatment of the sample, such as, for example, drying agents for the purpose of the preservation of the sample, enzymes, protease inhibitors, as well as DNA fragments, RNA fragments, or agents and enzymes for initiating a nucleic amplification, . . . . These chemical agents can be provided using a matrix, such as a paper strip that is impregnated with reagents. These matrices can be placed in the very interior of the sample container, thus dispensing with adding other reaction agents to the sample container. Such matrices are available, for example, from the Whatman Company, under the name FTA® paper, which are described with the associated technologies, in the U.S. Pat. No. 5,496,562, U.S. Pat. No. 5,756,126, U.S. Pat. No. 5,807,527, U.S. Pat. No. 5,972,386 and US 5,985,327, incorporated here in their entirety by reference.

In certain variant embodiments, the head can also contain—in the hollow part that receives the sample—chemical agents for the subsequent treatment of the sample. These agents are described above and can be provided in addition to the agents that are already present in the sample container or at the site of the latter. They can also be provided in matrix form, as described above.

In a preferred embodiment of the device according to the invention, the protection tube has an external shape that is identical to that of a standard laboratory tube. Such a protection tube can be, for example, a standard plastic laboratory tube such as an Eppendorf tube, or a Steiner or Sorvall centrifuging tube. This invention thus makes possible an easy manipulation of the samples of biological material that were taken and does not require sophisticated or very specialized laboratory equipment, but can adapt to existing laboratory equipment, already commonly used in the laboratories for the treatment of samples.

For the implementation of the ear tag according to the invention, the procedure is performed as for the application of a standard ear tag on an animal, whereby taking the sample of tissue is carried out concomitantly without an additional operation. The procedure is followed using a clamp that is designed for this purpose according to the stages that consist in:
Affixing the male part and the female part of an ear tag as defined above on the ear of an animal by making the stem that carries the head penetrate into the cartilage of the ear, until the head has totally passed through the tissues. In so doing, the cutting edge at the end of the head cuts a fragment of animal tissues into sections and brings it into the central space of the head. The head arrives at the first opening at the proximal end of said sample container and is inserted perfectly into said first opening after having collected said fragment of tissues that constitutes a sample of biological material and that is now found in said sample container. The sample container is then detached from said ear tag, and the device for preservation of the biological sample is recovered.
Introducing said sample container into a protection tube, so as to protect said sample, and storing it under suitable conditions.

Optionally, it is possible to initiate additional manipulations of the sample. If necessary, the sample contents will be withdrawn from the protection tube so as to have access to said second opening, and one of the next operations will be initiated inside the sample container: addition of reagents for a chemical treatment, treatment that may or may not be in solution, dissolution, drying, heat treatment or nucleic amplification.

The ear tag that comprises a male part and a female part can comprise means of identification written on the male part, on the female part, or on both. If these identification means are found on the male part, they are then preferably located on the base. If they are on the female part, the former is then preferably placed on one plate that bears these means of identification. All of these identification means find their correspondence with the identifiers that are printed on the sample container to make possible a cross-reference between the tags and the samples, and thus there is the connection between a given sample and the animal from which it was taken. Additionally, the female part can use a holding ring, as indicated in, for example, EP 0913083.

The embodiment that is described below in detail will make it possible to better understand the invention, without, however, reducing the scope in some manner.

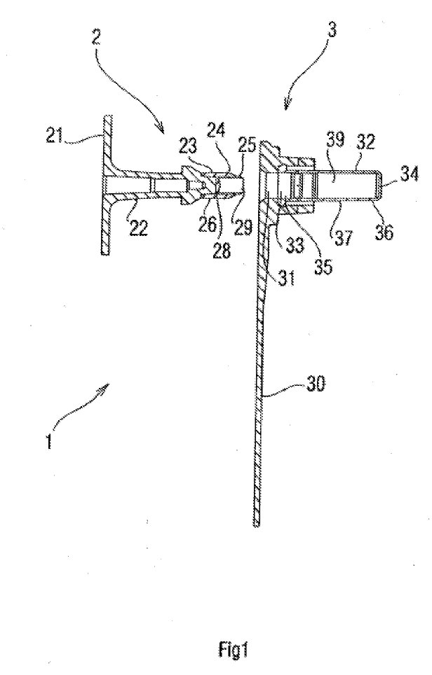

FIG. 1 shows in cutaway a marking ear tag according to the invention, before the assembly of the male and female parts.

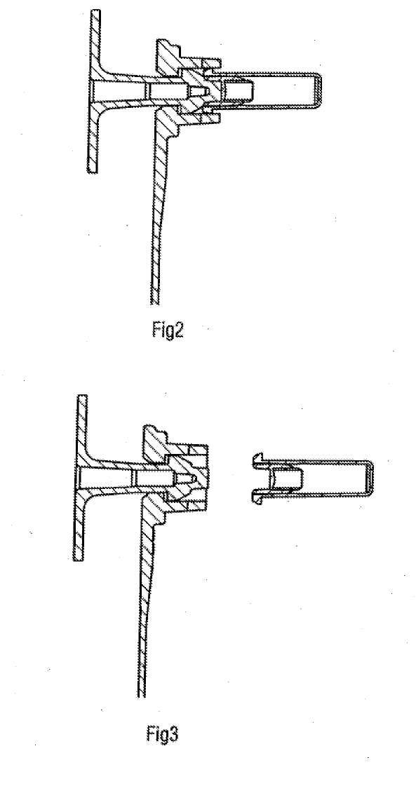

FIGS. 2 and 3 show in cutaway the same tag at different stages of the assembly of the male and female parts.

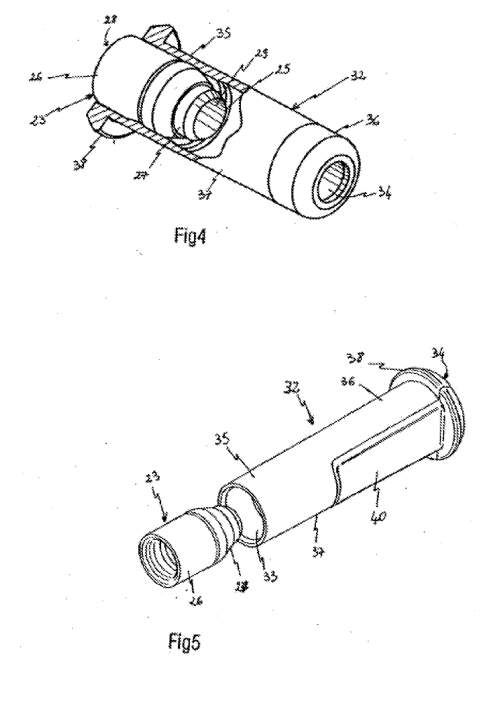

FIG. 4 is a depiction in cutaway perspective that shows the details of a sample container that is assembled with the head, with a proximal collar.

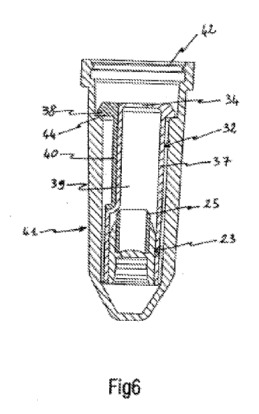

FIG. 5 is a depiction in exploded perspective of a sample container with a lateral flat surface and a distal collar.

FIG. 6 shows in cutaway an assembled sample container with the head in a protection tube.

In this example, and with reference to the drawings, the tag 1 comprises the male part 2 and the female part 3, designed to be assembled by irreversible interlocking on both sides of the ears of an animal.

The male part 2 comprises the base 1 and the stem 22 that constitutes an extension of said base. The stem 22 has the head 23 that is detachable, and whose distal part 27 is hollow. The head 23 is equipped with a cutting edge 25 that can select a sample of biological material when said head perforates the animal's ear. Here, the head 23 assumes the shape of a cylindrical tube whose proximal part 26 is sealed by the base 28 and whose distal part 27, which forms the cutting edge 25, comprises the open orifice 29. The cutting edge 25 is also circular. There is thus a blind bore within the cutting part that is used to collect the sample when the head passes through the animal's ear.

The female part 3 comprises a receiving element of the stem 22, and the marking plate 30 that is designed to constitute an identification tag. The central tube 31 is able to receive the head 23 and partially the stem 22 during the application of the ear tag 1. The tube 31 is combined with the sample container 32, of a general cylindrical shape, which can comprise a flat surface 40. The sample container 32 is made of polymethyl methacrylate (Perspex®). It is transparent at its side walls 37. It is placed in the axial extension of the tube 31. The container 32 has, at its proximal end 35, the first opening 33 that is able to receive the head 23 of the stem 22 during the application of the ear tag 1. At its distal end 36, the container 32 comprises the second opening 34 that provides access to the inside space 39 of said sample container.

The orifice 29 of the distal part 27 of the head 23 is placed in coaxial position opposite the central tube 31 and also opposite the sample container 32. The second opening 34 of the sample container 32 and the orifice 29 have an approximately identical surface. The inside wall of the proximal end 35 of the container 32 and the outside wall of the distal part 27 of the head 23 have a complementary shape such that said proximal end of the container and said distal part of the head are able to fit together perfectly, as can be seen in, for example, FIGS. 2 and 3.

The sample container 32 can be detached from the female part 3. Once the head 23 has been introduced into the first opening 33 of the sample container 32, the unit that consists of said head and said sample container is detached from the ear tag 1 that has just been applied on the ear and constitutes a device for preservation of the sample according to the invention, which can be stored and later subjected to treatment.

The container 32 comprises the collar 38 that constitutes a means for attachment of said container to a protection tube 41. It is located on the outside wall of the container 32 at its proximal end (FIG. 4) or distal end (FIG. 5). It holds the container 32 with the protection tube 41 by suspension at rest on the shoulder 44 that is located on the inside wall of the tube 41, which makes possible a perfectly hermetic protection of the sample container inside the tube.

FIGS. 1, 2 and 3 illustrate different stages for the application of an ear tag during which the head 23 and the sample container 32 are assembled and then ejected from the latter.

FIG. 6 illustrates the method for preserving a sample of tissues using the device that is described above, comprising the protection tube 41, of the Eppendorf tube type, closed by its cover 42, containing the container 32 at rest on the shoulder 44, with the head 23 in which the sample (not shown) and optionally chemical or biological compounds (not shown) are housed.

The different elements of this invention included in the description, the claims and/or the attached drawings may be important, by themselves or combined with one another, for producing the various forms of the reaction.

1. Ear tag (1) for identifying an animal, comprising a male part (2) and a female part (3) that are designed to be assembled by irreversible interlocking on both sides of said animal's ear,
Whereby said male part comprises a base (21) and a stem (22) constituting an extension of said base, said stem having a removable head (23) whose distal part (27) is hollow and is equipped with a cutting edge (25) that can cut a sample of biological material into sections when said head perforates the animal's ear,
Whereby said female part comprises a central tube (31) that can receive said head and at least partially said stem during the application of the ear tag, whereby said tube is combined with a detachable sample container (32) that is placed in the axial extension of said tube, said container having at its proximal end (35) a first opening (33) that can receive the head (23) of said stem during the application of the ear tag, characterized in that said container comprises at its distal end (36) a second opening (34) that provides access to the inside space of said sample container.

2. Tag according to claim 1, wherein the head (23) assumes the shape of a capsule whose proximal part (26) is sealed by a base (28) and whose distal part (27) that is equipped with said cutting edge is an open orifice (29).

3. Tag according to claim 1, wherein the orifice (29) of the distal part (27) of the head (23) is placed in the same axis as the sample container (32).

4. Tag according to claim 1, wherein the second opening (34) of the sample container (32) and the orifice (29) of the distal part (27) of the head (23) have an identical or approximately identical surface.

5. Tag according to claim 1, wherein the second opening (34) of the sample container (32) has a surface that is inferior to that of the orifice (29) of the distal part (27) of the head (23).

6. Tag according to claim 1, wherein the inside wall of the proximal end (35) of the container (32) and the outside wall of the distal part (27) of the head (23) have a complementary shape such that said proximal end of the container and said distal part of the head are able to fit together perfectly.

7. Tag according to claim 1, wherein the head (23) assumes the shape of a cylindrical capsule, and the cutting edge (25) is circular.

8. Tag according to claim 1, wherein the sample container (32) assumes the general shape of a cylinder that comprises said first opening (33) at its proximal end (35) and said second opening (34) at its distal end (36).

9. Tag according to claim 1, wherein the outside wall of the sample container (32) comprises a flat surface (40) that can receive or be used as substrate for an identifying marking.

10. Tag according to claim 1, wherein the sample container (32) is transparent at least at its side walls (37).

11. Tag according to claim 1, wherein the sample container (32) is made of one or more plastic materials that are selected from the group that comprises polymethyl methacrylate, polycarbonate, polypropylene, polyethylene, polymethylpentene, terephthalic polyethylene, polystyrene, polyvinyl chloride, and polyvinyl toluene.

12. Device for preservation of a biological sample taken from an animal's ear during the application of an identification tag according to claim 1, wherein it comprises:
i) A sample container (32) that comprises, at its proximal end (35), a first opening (33) that is sealed by the distal part of a hollow head (23) that contains said biological sample, and, at its distal end (36), a second opening (34) that provides access to the inside space of said sample container, whereby said proximal end of the container and said distal part of the head fit together perfectly, and
ii) A protection tube (41) that is equipped with a cover (42) that is designed to seal the neck (43) of said tube, whereby said tube is able to preserve the sample container (32) in a closed space.

13. Device according to claim 12, wherein it comprises means for holding the sample container (32) in suspension at an intermediate height in the protection tube (41).

14. Device according to claim 13, wherein said holding means comprise a collar (38) that is located on the outside wall of the container (32) and a shoulder (44) that is located on the inside wall of the protection tube (41), whereby said collar rests on said shoulder.

15. Device according to claim 14, wherein the collar (38) is located at the distal end (36) of the outside wall (37) of the sample container (32).

16. Device according to claim 12, wherein the protection tube (41) contains at least a first chemical or biochemical compound that is useful for the treatment of the sample.

17. Device according to claim 12, wherein the hollow head (23) contains at least a second chemical or biochemical compound that is useful for the treatment of the sample.

18. Device according to claim 16, wherein said at least one first and second chemical or biochemical compounds are selected from among the drying agents, the protease inhibitors, the enzymes, the DNA fragments, and the RNA fragments.

19. Device according to claim 12, wherein the protection tube (41) has an external shape that is identical to that of a standard laboratory tube.
